(19) United States
(12) Patent Application Publication
(10) Pub. No.: US 2012/0123353 A1
(43) Pub. Date: May 17, 2012

(54) An Ophthalmic Device and an Intravitreal Method

(75) Inventors: Marc August Hilde Veckeneer, Dordrecht (NL); Gerrit Jan Vijfvinkel, Gecrvliet (NL)

(73) Assignee: D.O.R.C. Dutch Ophthalmic Research Center (International) B.V.

(21) Appl. No.: 13/142,479

(22) PCT Filed: Dec. 28, 2009
(86) PCT No.: PCT/NL2009/050808

§ 371 Date: Dec. 21, 2011

(30) Foreign Application Priority Data
Dec. 29, 2008 (NL) 2002379

(51) Int. Cl. A61M 5/32

(52) U.S. Cl. 604/272

(57) ABSTRACT

The invention relates to an ophthalmic device for intravitreal administration of a substance into a human or animal eye by means of a needle. The device comprises a support structure having a lower surface for contacting the conjunctiva of the sclera, the lower surface being provided with an aperture for receiving the needle. The support structure further comprises a substantially tapering part directed away from the lower surface. In addition, the device comprises a channel provided in the tapering part wherein a first end of the channel forms the aperture in the lower surface.

The invention relates to an ophthalmic device for intravitreal administration of a substance into a human or animal eye by means of a needle, comprising a support structure having a lower surface for contacting the conjunctiva of the sclera, the lower surface being provided with an aperture for receiving the needle.

In ophthalmology, a medical substance can be injected into the corpus vitreum to treat eye diseases such as intraocular inflammation, macular edema, retinal new vessel formation as in proliferative diabetic retinopathy and choroidal neovascularisation as in age-related macular degeneration. The injection is performed by a needle that is usually pierced through the sclera at the so-called pars plana at a distance of approximately 3.5 mm from the limbus being the corneoscleral transition zone.

The position where the needle is to be inserted into the sclera is usually determined on the basis of the ophthalmologist's visual assessment and the aid of a measuring device. A needle that is inserted at an incorrect position or at an incorrect angle may cause complications, such as intraocular hemorrhage, lens damage leading to cataract, retinal perforation and detachment. Further, eyelids are kept open by separate fixation elements to avoid that a blinking movement of the patient's eye interferes with the insertion of the needle. However, the application of such eyelid fixation elements is uncomfortable for the patient.

It is an object of the invention to provide an ophthalmic device according to the preamble wherein at least one of the disadvantages mentioned above is counteracted. In particular, the invention aims at providing an ophthalmic device according to the preamble that increases the safety, the surgical simplicity and/or the patient comfort of the intravitreal injection procedure. Thereto, the support structure further comprises a substantially tapering part directed away from the lower surface and the device also comprises a channel provided in the tapering part wherein a first end of the channel forms the aperture in the lower surface.

By providing the tapered part including the channel, contact of the eyelid margin with the injection site is avoided. As a result, the device according to the invention provides an antiseptic effect. A further antiseptic effect may be obtained by guiding droplets, such as a disinfectant, e.g. povidon iodine, through the channel towards the injection site, thus increasing the safety of the injection procedure. Obviously, also other droplets, such as an anesthetic, e.g. cocaine, can be guided through the channel. Optionally, the droplets are fed to the conjunctiva via a passage formed by the second end of the channel, towards a second substantially tapering part serving as a receptor, both tapering parts being oriented in an opposite way forming an hour-glass shape.

It is further noted that the channel structure guides the needle through the device into the eye in a predetermined orientation, preferably perpendicular with respect to the lower surface of the support structure, thereby preventing damage caused by poor orientation of the needle. In addition, the channel structure enables setting a predetermined maximum penetration depth of the needle, e.g. 5 mm into the vitreous body. To that end, an appropriate needle and channel geometry and dimensions can be chosen to set the maximum penetration depth accurately, thereby preventing undesired extremely deep penetration of the needle into the eye.

The all-in-one design of the ophthalmic device according to the invention further simplifies the procedure in such a way that the skills required to perform the intravitreal procedure are greatly reduced. To precisely identify the injection site which is at the pars plana, the size of the lower surface may act as a measuring device. Thereto, the distance between the needle guiding channel aperture and the edge of the support structure lower surface can be chosen to have a predetermined distance, e.g. exactly 3.5 mm. When the lower surface edge is placed on the sclera, tangent to the limbus, the needle channel being directed away from the cornea towards the conjunctival fornix, the penetration site will be precisely through the pars plana.

Further, the all-in-one design of the device according to the invention renders the need to insert an eyelid speculum superfluous since there is no need to grasp the conjunctiva with forceps. The lower part of the device prevents contact of eyelids with the injection site or needle as the surface of the circumference of the lower part of the device that will make contact with the conjunctiva, is serrated. The device being controlled through a hand piece can thus be used to stabilize and control the eyeball.

Moreover, by providing the tapered part with the channel, a structure is obtained that provides a relatively comfortable and effective protection against the eyelids when the needle is received in the aperture. Here, the eyelids can contact the tapered part in an ergonomic way without reaching the needle and the penetration site. Further, due to the presence of the channel, the needle may be inserted towards the aperture of the lower surface, even when the eyelids are in semi-closed position.

In an advantageous embodiment according to the invention, the support structure further comprises a second substantially tapering part, wherein the tapering parts are oriented in an opposite way forming an hour-glass or diabolo shape. By providing the second substantially tapering part, the needle can easily and safely be guided towards the channel in the first substantially tapering part. In principle, the device can also be implemented without the second substantially tapering part, e.g. in order to obtain a very compact device.

In a further advantageous embodiment according to the invention, the needle forms an integral member of the device so that operation of the device can further be simplified.

The invention also relates to an intravitreal method of administrating a substance into a human or animal eye.

Other advantageous embodiments according to the invention are described in the following claims.

By way of example only, embodiments of the present invention will now be described with reference to the accompanying figures in which

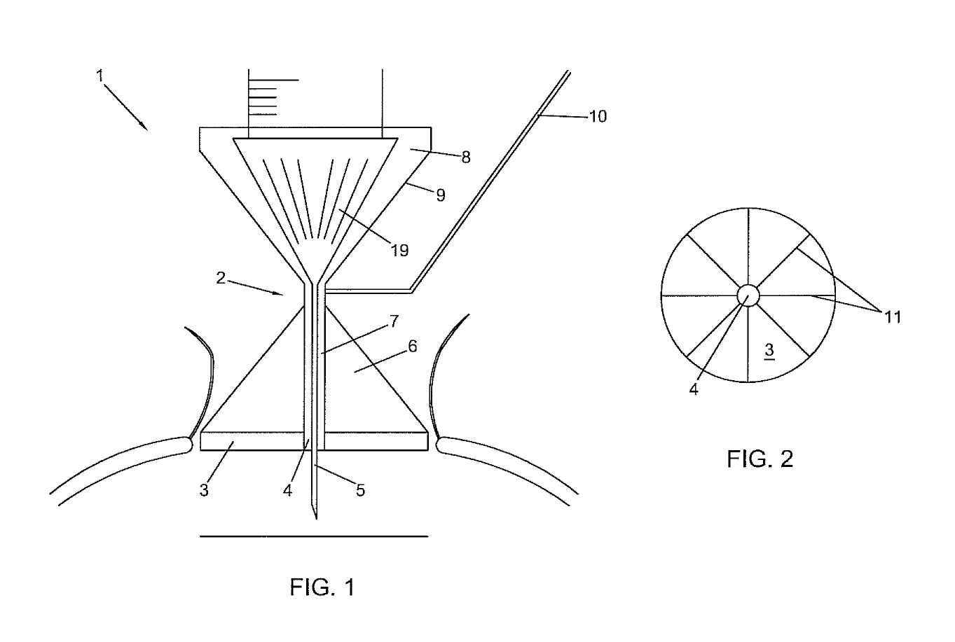

FIG. 1 shows a schematic cross sectional view of a first embodiment of an ophthalmic device according to the invention;

FIG. 2 shows a schematic bottom view of the ophthalmic device of FIG. 1;

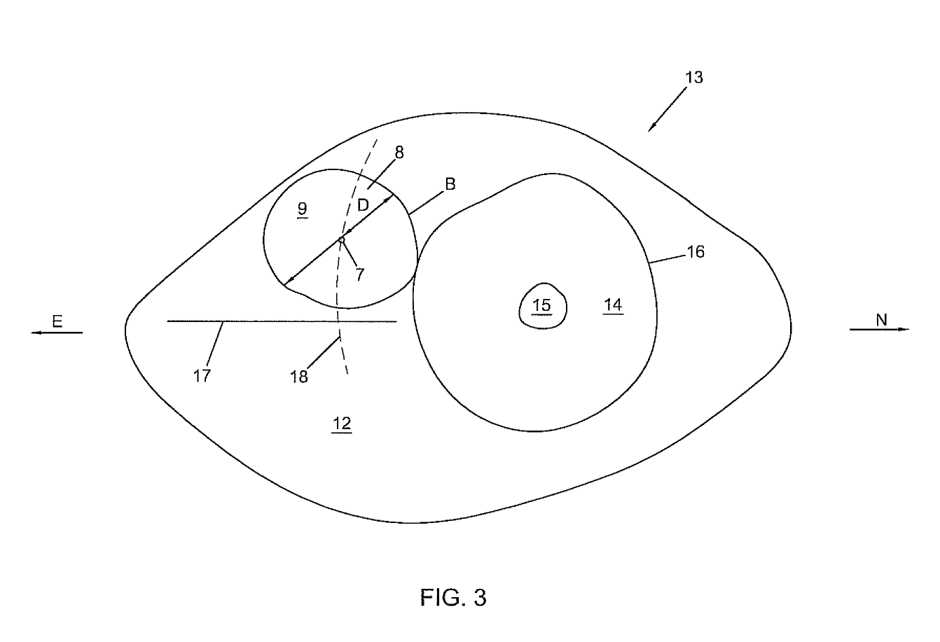

FIG. 3 shows a schematic top view of the ophthalmic device of FIG. 1 when positioned on the sclera;

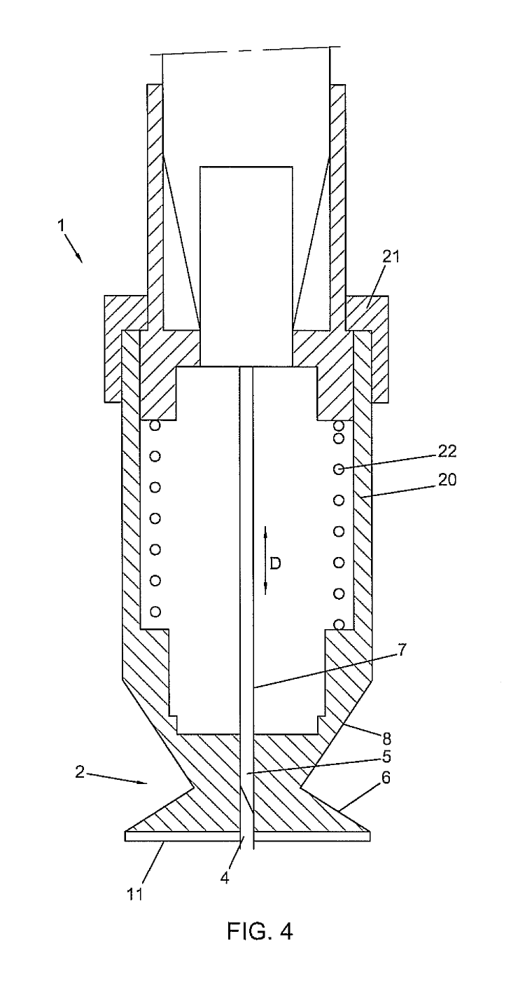

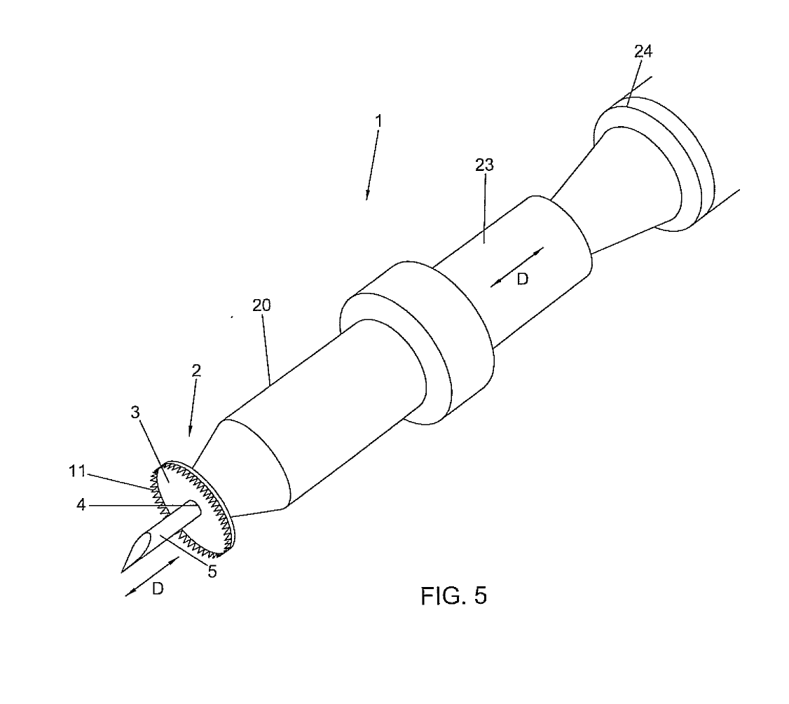

FIG. 4 shows a schematic cross sectional view of a second embodiment of an ophthalmic device according to the invention;

FIG. 5 shows a schematic perspective view of the ophthalmic device of FIG. 4; and

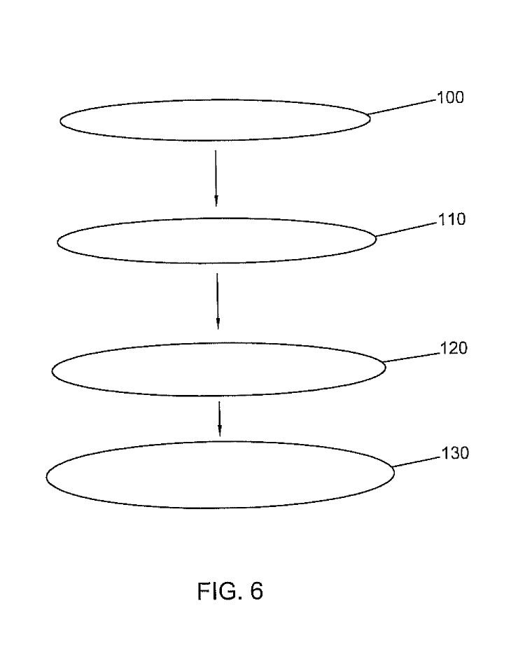

FIG. 6 shows a flowchart of method steps according to the invention.

It is noted that the figures show merely a preferred embodiment according to the invention. In the figures, the same reference numbers refer to equal or corresponding parts.

FIG. 1 shows a schematic cross sectional view of an ophthalmic device 1 according to the invention. The device 1 comprises a support structure 2 having a lower surface 3 for contacting the conjunctiva of a patient's eye. The lower surface 3 is provided with an aperture 4 for receiving a needle 5. Further, the support structure 2 comprises a substantially tapering part 6 directed away from the lower surface 3. The structure 2 also comprises a substantially straight channel 7 provided in the tapering part. The orientation of the channel 7 is perpendicular to the lower surface 3 that makes contact with the eye in order to secure that the needle penetrates the sclera perpendicularly. However, also other orientations can be applied, e.g. a somewhat tilted orientation with respect to the normal of the lower surface 3. In addition, the support structure 2 comprises a second substantially tapering part 8, the tapering parts being oriented in an opposite way forming an hour-glass shape. The hour-glass shaped is thus formed by two conical ends that are directed to each other to form a diabolo shape. Here, a first end of the channel 7 forms the aperture 4 in the lower surface 3, while a second end of the channel 7 forms a passage to an inner surface 9 of the second tapering part 8. The inner surface of the second tapering part is substantially funnel shaped, thus facilitating feeding anesthetic droplets and/or disinfectant droplets to flow towards the aperture 4 of the lower surface 3. The ophthalmic device 1 further comprises a grip 10 attached to the support structure 2 so as to handle the device 1.

FIG. 2 shows a schematic bottom view of the ophthalmic device 1. The lower surface 3 of the support structure 2 is optionally provided with gripping elements for gripping the sclera. In the shown embodiment, the gripping elements comprise a number of ribs 11 extending in a radial direction so that surgeon can easily grip the conjunctiva of the sclera. Obviously, also other gripping element types can be applied, such as a serrated or corrugated formed lower surface 3 of the support structure 2.

FIG. 3 shows a schematic top view of the ophthalmic device 1 of FIG. 1 when positioned on the sclera 12 of a patient's right eye 13. In

FIG. 3 the nose direction N and the ear direction E are indicated. Further, the cornea 14 is shown enclosing the pupil 15. The so-called limbus 16 circumvents the cornea 14.

FIG. 4 shows a schematic cross sectional view of a second embodiment of an ophthalmic device 1 according to the invention. Here, the second end of the channel 7 does not form a passage to an inner surface of the second tapering part, but extends in a housing 20 that is connected to the second tapering part 8 and receives the needle 5, so that the needle 5 is integrated with the device 1. Since, in the embodiment shown in FIG. 4, the needle 5 and the device 1 are not separate elements, but form a single integrated unit, operation of the device 1 can further be simplified. As a consequence, the skills required to perform the intravitreal procedure are further reduced, as the device 1 can in principle be operated by a single hand.

The needle 5 is movable in a pre-determined range in a direction D through the channel 7, along the axis of the channel 7. In particular, the movement of the needle 5 is stopped at two end positions of the pre-determined range by one or more stoppers to control the movements of the needle 5. Preferably, in a retracted position, the needle does not extend through the aperture 4 to avoid damage or injuries. Further, in a distal position, the needle may penetrate through the sclera 12. A stopper can e.g. be implemented by providing the needle 5 with an integrated member 21 that abuts against the housing 20 when the pre-determined distal position has been reached. Similarly, a pre-defined retracted position can be defined. However, the pre-defined range can also be open at the retracted position, thereby allowing that the needle 5 is removed from the housing 20 of the device 1 relatively easily.

The device 1 further comprises an elastic element 22, e.g. implemented as a spring element, for exerting a backward pulling force on the needle 5 in a direction away from the lower surface 3 of the support structure 2, so as to obtain a device that normally has a retracted needle 5 when no manual pulling forces are exerted on the needle 5, thereby further enhancing the safety of the device 1. In FIG. 4 the device is depicted when the needle 5 is in the retracted position.

FIG. 5 shows a schematic perspective view of the ophthalmic device 1 shown in FIG. 4. Here, the needle is depicted in a distal position extending through the aperture 4. The needle 5 is connected to a hub 23 having a luer lok connection module 24 to allow easy disconnection with a tube containing substance to be administrated to the human or animal eye. Obviously, also other connection modules 24 can be applied to the needle hub 23, such as another screw connection or connection modules based on another connection mechanism, such as a click mechanism.

FIG. 6 shows a flowchart of method steps according to the invention. During application of the device 1, an intravitreal method of administrating a substance into a human or animal eye 11 can be facilitated. The method comprises a placing step 100 of placing the support structure 2 on the conjunctiva of the sclera 12 such that the lower surface 3 of the support structure 2 contacts the conjunctiva of the sclera and a boundary B of the lower surface 2 is tangent to the limbus 16, such that the lower surface 2 is positioned above or below a middle line 17 of the eye in order to avoid damaging nerves in the eye 13. The diameter D of the lower surface 3 is 8 mm, so that the aperture 7 is above the pars plana 18 of the eye 13. After placing the support structure 2 on the eye 13, anesthetic droplets and/or disinfectant droplets can be inserted into the channel 7. Then, the method comprises an inserting step 110 of inserting a needle through the substantially straight channel 7 provided in the first substantially tapering part 6. The channel 7 is wide enough for receiving a needle having a diameter of at least 27 gauge. In principle, also other channel diameter can be applied. Further, the method comprises a piercing step 120 of piercing the needle 5 through the sclera 12 and a injecting step 130 of pressurizing a substance, such as a medical substance, via the needle 5 into the corpus vitreum. When employing the device as shown in FIGS. 4 and 5 the piercing step 120 comprises the step of moving the needle 5 against a backward pulling force that is exerted by an elastic element 22 on the needle 5 in a direction away from the lower surface 3 of the support structure 2. The needle 5 is provided with a stopper 19 that may be implemented in various ways to control a penetration depth of the needle 5 during the piercing movement in order to avoid damage in the eye. The stopper can e.g. be implemented as a top section having a wider dimension than the diameter of the channel 7, as shown in FIG. 1.

The ophthalmic device 1 according to the invention therefore facilitates keeping the eye open, fixing the sclera, determining the pars plana and piercing the needle using a single instrument.

The invention is not restricted to the embodiments described herein. It will be understood that many variants are possible.

Other such variants will be obvious for the person skilled in the art and are considered to lie within the scope of the invention as formulated in the following claims.

1. An ophthalmic device for intravitreal administration of a substance into a human or animal eye by means of a needle, comprising a support structure having a lower surface for contacting the conjunctiva of the sclera, the lower surface being provided with an aperture for receiving the needle, wherein the support structure further comprises a substantially tapering part directed away from the lower surface, further comprising a channel provided in the tapering part wherein a first end of the channel forms the aperture in the lower surface.

2. The ophthalmic device according to claim 1, wherein the support structure further comprises a second substantially tapering part, the tapering parts being oriented in an opposite way forming an hour-glass shape.

3. The ophthalmic device according to claim 2, wherein a second end of the channel forms a passage to an inner surface of the second tapering part.

4. The ophthalmic device according to claim 2, wherein the inner surface of the second tapering part is substantially funnel shaped.

5. The ophthalmic device according to claim 1, further comprising a grip attached to the support structure.

6. The ophthalmic device according to claim 1, wherein the needle forms an integral member of the device.

7. The ophthalmic device according to claim 6, wherein the needle is connected to a hub having a connection module provided with a luer lok connector part for connection with a tube containing substance to be administrated to the human or animal eye.

8. The ophthalmic device according to claim 6, wherein the needle is movable in a pre-determined range in a direction through the channel.

9. The ophthalmic device according to claim 8, further comprising an elastic element for exerting a backward pulling force on the needle in a direction away from the lower surface of the support structure.

10. The ophthalmic device according to claim 1, wherein the lower surface of the support structure is provided with gripping elements for gripping the sclera.

11. The ophthalmic device according to claim 1, wherein the channel is wide enough for receiving a needle having a diameter of at least 27 gauge.

12. An intravitreal method of administrating a substance into a human or animal eye, comprising placing a support structure on the sclera such that a lower surface of the support structure contacts the conjunctiva of the sclera and a boundary of the lower surface is tangent to the limbus, further comprising inserting a needle through a channel provided in a substantially tapering part directed away from the lower surface of the support structure, a first end of the channel forming an aperture in the lower surface, wherein the method also comprises piercing the needle through the sclera and pressurizing the substance via the needle into the eye.

13. The method according to claim 12, wherein piercing through the sclera comprises the step of moving the needle against a backward pulling force that is exerted by an elastic element on the needle in a direction away from the lower surface of the support structure.
